(19) United States
(12) Patent Application Publication
(10) Pub. No.: US 2023/0115958 A1
(43) Pub. Date: Apr. 13, 2023

(54) INFORMATION DISCLOSURE SYSTEM, SERVER, AND INFORMATION DISCLOSURE METHOD

(71) Applicant: CureApp, Inc., Tokyo (JP)

(72) Inventor: Yutaka Kohada, Tokyo (JP)

(21) Appl. No.: 18/078,870

(22) Filed: Dec. 9, 2022

Related U.S. Application Data
(63) Continuation of application No. PCT/JP2021/015172, filed on Apr. 12, 2021.

(30) Foreign Application Priority Data
Jun. 12, 2020 (JP) 2020-102655

(51) Int. Cl. G16H 10/60; G06F 21/62

(52) U.S. Cl. CPC G16H 10/60; G06F 21/6245

(57) ABSTRACT

In an information disclosure system, when a selection of details to be disclosed to a medical worker out of patient information including one or more types of details pertaining to treatment of a patient is received by a patient terminal, an information disclosure authorization server receives request information for requesting disclosure of the selected details transmitted from the patient terminal, the information disclosure authorization server generates authorization information for authorizing disclosure of details included in the patient information based on the received request information and provides the generated authorization information to the patient terminal owned by the patient, and the information disclosure authorization server authorizes disclosure of the details included in the patient information of which the selection has been received by the patient terminal based on the authorization information and enables acquisition of the patient information from the storage unit that stores the details of the patient information when the authorization information provided to the patient terminal is received by the medical worker terminal and the authorization information transmitted from the medical worker terminal is received by the information disclosure authorization server.

### INCORPORATION BY REFERENCE

This application is a continuation International Patent Application No. PCT/JP2021/015172 filed on Apr. 12, 2021, which claims the benefit of priority to Japanese Patent Application No. 2020-102655 filed Jun. 12, 2020, the contents of which are incorporated herein by reference in their entireties.

### TECHNICAL FIELD

This disclosure relates to an information disclosure system, a server, and an information disclosure method.

### BACKGROUND

Techniques of requesting a patient's approval when medical records of the patient are disclosed are known. In the technique described in Japanese Unexamined Patent Application Publication No. 2007-213139, authentication of a patient is requested when patient information including personal information of the patient and medical records and examination records of the patient is downloaded from a storage unit, and authentication of a medical worker and a patient is requested when additional patient information is uploaded to the storage unit.

Recently, therapeutic applications prescribed by medical workers have been installed on patient terminals owned by patients. In that instance, details acquired by such a therapeutic application may be requested by a third party other than a medical institution to which a medical worker having prescribed the therapeutic application belongs.

Patient information corresponds to “consideration-required personal information” in the Japanese Personal Information Protection Law. As described in Paragraph 2 of Article 17 of the Personal Information Protection Law, approval of a principal or substitute of a patient is required for disclosure of patient information and thus the patient needs to authorize disclosure of the patient information to a person who has explicitly requested the disclosure. Accordingly, there is a need for a structure (system) that can allow a medical institution to acquire patient information on the basis of explicit approval of the patient.

It could therefore be helpful to provide an information disclosure system, a server, and an information disclosure method that can protect disclosure of details acquired using a patient terminal.

### SUMMARY

I thus provide an information disclosure system including a patient terminal that is used by a patient, an information disclosure authorization server that authorizes information disclosure, and a medical worker terminal that is used by a medical worker and that includes an output unit, the information disclosure system including: a selection receiving unit provided in the patient terminal and configured to receive a selection of details to be disclosed to the medical worker out of patient information including one or more types of details pertaining to treatment of the patient; a request unit provided in the patient terminal and configured to generate request information for requesting disclosure of the details of which the selection is received by the selection receiving unit; an authorization unit provided in the information disclosure authorization server and configured to generate authorization information for authorizing disclosure of details included in the patient information on the basis of the request information generated by the request unit and to provide the generated authorization information to the patient terminal owned by the patient; an input receiving unit provided in the medical worker terminal and configured to receive an input of the authorization information when the authorization information provided from the authorization unit to the patient terminal is received; an authentication unit provided in the information disclosure authorization server and configured to authorize disclosure of details included in the patient information of which the selection is received by the selection receiving unit on the basis of the authorization information received by the input receiving unit and to enable acquisition of the details of the patient information; and an output control unit provided in the medical worker terminal and configured to control the output unit such that the patient information is output when acquisition of the patient information is authorized by the authentication unit.

The information disclosure system may further include a storage unit configured to store patient information, the authentication unit may be configured to enable acquisition of the details of the patient information from the storage unit, and the output control unit may be configured to acquire the details of the patient information from the storage unit and to output the patient information.

I also provide an information disclosure system including a storage unit configured to store information, a patient terminal that is used by a patient, an information disclosure authorization server that authorizes information disclosure, and a medical worker terminal that is used by a medical worker and that includes an output unit, the information disclosure system including: the storage unit configured to store patient information including one or more types of details pertaining to treatment of the patient; a selection receiving unit configured to cause the medical worker terminal to receive a selection of details to be disclosed to the medical worker out of the patient information; a first request unit configured to cause the medical worker terminal to generate first request information for requesting disclosure of the details of which the selection is received by the selection receiving unit; a transmission unit configured to cause the information disclosure authorization server to generate request information for requesting the patient to disclose the patient information on the basis of the first request information generated by the first request unit and to transmit the generated request information to the patient terminal; a second request unit configured to cause the patient terminal to acquire the request information transmitted by the transmission unit, to generate second request information for requesting disclosure of the patient information in response to the request information, and to transmit the generated second request information to the information disclosure authorization server; an authorization unit configured to cause the information disclosure authorization server to generate authorization information for authorizing disclosure of details included in the patient information on the basis of the second request information generated by the second request unit and to provide the generated authorization information to the patient terminal owned by the patient; an input receiving unit configured to cause the medical worker terminal to receive an input of the authorization information when the authorization information provided from the authorization unit to the patient terminal is received; an authentication unit configured to cause the information disclosure authorization server to authorize disclosure of details included in the patient information of which the selection is received by the selection receiving unit on the basis of the authorization information received by the input receiving unit and to enable acquisition of the details of the patient information from the storage unit; and an output control unit configured to cause the medical worker terminal to acquire the details of the patient information from the storage unit and to control the output unit such that the patient information is output when acquisition of the patient information is authorized by the authentication unit.

The authorization unit may be configured to add deadline information pertaining to a validity term in which patient information is disclosable to the authorization information.

The patient terminal may transmit patient information to the storage unit when patient information which is input on the basis of the patient's operation and patient information which is generated by performing measurement of the patient using a sensor provided in the patient terminal are acquired.

The patient terminal may include a setting unit configured to set at least one of a plurality of types of details pertaining to medical treatment of a patient, actions of a patient, and a biological body of a patient as the patient information such that the at least one is disclosable to the medical worker on the basis of the patient's operation when the plurality of types of details are acquired.

I further provide a server connected to a patient terminal used by a patient and a medical worker terminal used by a medical worker via a communication network and that authorizes information disclosure, the server including: a communication unit configured to receive request information for requesting disclosure of selected details to be disclosed to the medical worker transmitted from the patient terminal when a selection of the details to be disclosed to the medical worker out of patient information including one or more types of details pertaining to treatment of the patient is received by the patient terminal; an authorization unit configured to generate authorization information for authorizing disclosure of the details included in the patient information on the basis of the request information received by the communication unit and to provide the generated authorization information to the patient terminal owned by the patient; and an authentication unit configured to authorize disclosure of details included in the patient information of which the selection is received by the patient terminal on the basis of the authorization information and to enable acquisition of the details of the patient information from a storage unit in which the details of the patient information are stored when the authorization information provided to the patient terminal is received by the medical worker terminal and the authorization information transmitted from the medical worker terminal is received by the communication unit.

I still further provide an information disclosure method performed by an information disclosure system including a patient terminal that is used by a patient, an information disclosure authorization server that authorizes information disclosure, and a medical worker terminal that is used by a medical worker and that includes an output unit, the information disclosure method including: a selection receiving step of causing the patient terminal to receive a selection of details to be disclosed to the medical worker out of patient information; a request step of causing the patient terminal to generate request information for requesting disclosure of the details of which the selection is received in the selection receiving step; an authorization step of causing the information disclosure authorization server to generate authorization information for authorizing disclosure of details included in the patient information on the basis of the request information generated in the request step and to provide the generated authorization information to the patient terminal owned by the patient; an input receiving step of causing the medical worker terminal to receive an input of the authorization information when the authorization information provided to the patient terminal in the authorization step is received; an authentication step of causing the information disclosure authorization server to authorize disclosure of details included in the patient information of which the selection is received in the selection receiving step on the basis of the authorization information received in the input receiving step and to enable acquisition of the details of the patient information; and an output control step of causing the medical worker terminal to acquire the details of the patient information and to control the output unit such that the patient information is output when acquisition of the patient information is authorized in the authentication step.

I yet further provide an information disclosure method performed by an information disclosure system including a storage unit configured to store information, a patient terminal that is used by a patient, an information disclosure authorization server that authorizes information disclosure, and a medical worker terminal that is used by a medical worker and that includes an output unit, the information disclosure method including: a storage step of causing the storage unit to store patient information including one or more types of details pertaining to treatment of the patient; a selection receiving step of causing the medical worker terminal to receive a selection of details to be disclosed to the medical worker out of the patient information; a first request step of causing the medical worker terminal to generate first request information for requesting disclosure of the details of which the selection is received in the selection receiving step; a transmission step of causing the information disclosure authorization server to generate request information for requesting the patient to disclose the patient information on the basis of the first request information generated in the first request step and to transmit the generated request information to the medical worker terminal; a second request step of causing the patient terminal to acquire the request information transmitted to the medical worker terminal in the transmission step, to generate second request information for requesting disclosure of the patient information in response to the request information, and to transmit the generated second request information to the information disclosure authorization server; an authorization step of causing the information disclosure authorization server to generate authorization information for authorizing disclosure of details included in the patient information on the basis of the second request information generated in the second request step and to provide the generated authorization information to the patient terminal owned by the patient; an input receiving step of causing the medical worker terminal to receive an input of the authorization information when the authorization information provided to the patient terminal in the authorization step is received; an authentication step of causing the information disclosure authorization server to authorize disclosure of details included in the patient information of which the selection is received in the selection receiving step on the basis of the authorization information received in the input receiving step and to enable acquisition of the details of the patient information from the storage unit; and an output control step of causing the medical worker terminal to acquire the details of the patient information stored in the storage step and to control the output unit such that the patient information is output when acquisition of the patient information is authorized in the authentication step.

When a selection of details to be disclosed to a medical worker out of patient information including one or more types of details pertaining to treatment of a patient is received by a patient terminal, the information disclosure authorization server receives request information for requesting disclosure of the details selected by the patient terminal, the information disclosure authorization server generates authorization information for authorizing disclosure of details included in the patient information on the basis of the received request information and provides the generated authorization information to the patient terminal owned by the patient, and the information disclosure authorization server authorizes disclosure of the details included in the patient information of which the selection has been received by the patient terminal on the basis of the authorization information and enables acquisition of the patient information from the storage unit that stores the details of the patient information when the authorization information provided to the patient terminal is received by the medical worker terminal and the authorization information transmitted from the medical worker terminal is received by the information disclosure authorization server. Accordingly, it is possible to perform protection pertaining to disclosure of details acquired by a patient terminal.

With the server and the information disclosure method, the same advantageous effects as in the information disclosure system can be achieved.

### BRIEF DESCRIPTION OF THE DRAWINGS

Features, advantages, and technical and industrial significance of examples will be described below with reference to the accompanying drawings, in which like numerals denote like elements, and wherein:

FIG. 1 is a diagram illustrating an information disclosure system according to an example.

FIG. 2 is a block diagram illustrating an information disclosure system according to a first example.

FIG. 3 is a diagram illustrating an example of a screen that is displayed on a display unit of a medical worker terminal to allow authorization information to be input.

FIG. 4 is a diagram illustrating an example of a screen that is displayed on a display unit of a medical worker terminal to acquire patient information.

FIG. 5 is a flowchart illustrating an information disclosure method according to the first example.

FIG. 6 is a block diagram illustrating an information disclosure system according to a second example.

FIG. 7 is a flowchart illustrating an information disclosure method according to the second example.

### REFERENCE SIGNS LIST

1 (1a, 1b) Information disclosure system
10 Storage unit
20 Patient terminal
22 Selection receiving unit
23 Setting unit
24 Request unit
30 Information disclosure authorization server
32 Authorization unit
33 Authentication unit
40 Medical worker terminal
42 Input receiving unit
43 Output control unit
100 Storage unit
200 Patient terminal
202 Second setting unit
203 Second request unit
300 Information disclosure authorization server
302 Authorization unit
303 Authentication unit
400 Medical worker terminal
402 Selection receiving unit
403 First request unit
404 First setting unit
405 Input receiving unit
406 Output control unit

### DETAILED DESCRIPTION

Hereinafter, examples of my systems, servers and methods will be described.

The word “information” can be exchanged with “data,” and the word “data” can be exchanged with “information.”

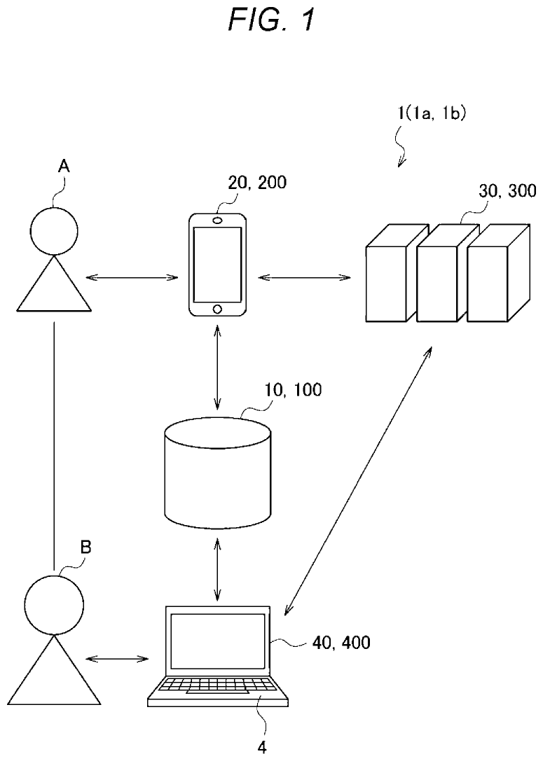

FIG. 1 is a diagram illustrating an information disclosure system 1 (1a or 1b) according to an example.

The information disclosure system 1 (1a or 1b) is a system that discloses medical information of a patient A to a medical worker B. The information disclosure system 1 (1a or 1b) includes a storage unit 10 or 100, a patient terminal 20 or 200, an information disclosure authorization server 30 or 300, and a medical worker terminal 40 or 400.

For example, an application (for example, a therapeutic application, a medical application, or a biological application) is installed on the patient terminal 20, 200 owned by the patient A. For example, the patient A writes patient information on medicine and registers the patient information in the application. For example, the application handles, as patient information, information of recognition (points of view and values, for example, “a cigarette relieves stress”), information of actions (for example, the number of steps and the number of smoked cigarettes), biological information (for example, a carbon monoxide concentration), disease information (for example, diabetes), medicine ingestion history information (for example, an ingestion history of smoking-cessation aids), attribute information (for example, age), and disease consciousness information (for example, feeling suffocated). For example, in the application, a plurality of types of details such as a blood pressure, a heart rate, and a pulse rate of the patient A are registered as patient information by the patient A. Alternatively, for example, the application may acquire biological information (for example, a blood pressure) measured using various sensors (not illustrated) and register the biological information in corresponding details as patient information. Various types of applications may be divisionally used depending on symptoms of the patient A. When a plurality of applications are installed on the patient terminal 20 or 200, there may be details shared by the plurality of applications. The patient terminal 20 or 200 may store the details registered in the application in the storage unit 10 or 100.

Alternatively, the application may be stored in the storage unit 10 or 100, an image based on the application may be displayed on the patient terminal 20 or 200 which is connected to the storage unit 10 or 100 via the communication network, and patient information may be input and registered in the application.

Alternatively, the application may be stored in the information disclosure authorization server 30 or 300, an image based on the application may be displayed on the patient terminal 20 or 200 which is connected to the information disclosure authorization server 30 or 300 via the communication network, and patient information may be stored in the storage unit 10 or 100 when the patient information is input to and registered in the patient terminal 20 or 200.

When a request for disclosing patient information of a specific patient A stored in the storage unit 10 or 100 to a medical worker B is issued, the information disclosure authorization server 30 or 300 transmits to the patient A (patient terminal 20 or 200) ascertainment information for requesting ascertainment of whether the patient information is to be disclosed. When the patient A authorizes disclosure of the patient information, the information disclosure authorization server 30 or 300 transmits to the patient terminal 20 or 200 authorization information for authorizing disclosure of the patient information to the medical worker B.

The medical worker B acquires authorization information from the patient A (the patient terminal 20 or 200) and inputs the authorization information to the medical worker terminal 40 or 400. The medical worker terminal 40 or 400 transmits the input authorization information to the information disclosure authorization server 30 or 300 and acquires authentication for acquiring patient information from the information disclosure authorization server 30 or 300. The medical worker terminal 40 or 400 accesses the storage unit 10 or 100 on the basis of the acquired authentication, and the storage unit 10 or 100 acquires the patient information of the patient A by causing the information disclosure authorization server 30 or 300 to check whether the acquired authentication information is correct, and when it is correct, providing the patient information of the patient A to the medical worker terminal 40 or 400.

The information disclosure system 1 (1a or 1b) will be described below in detail.

### First Example

A first example will be first described below.

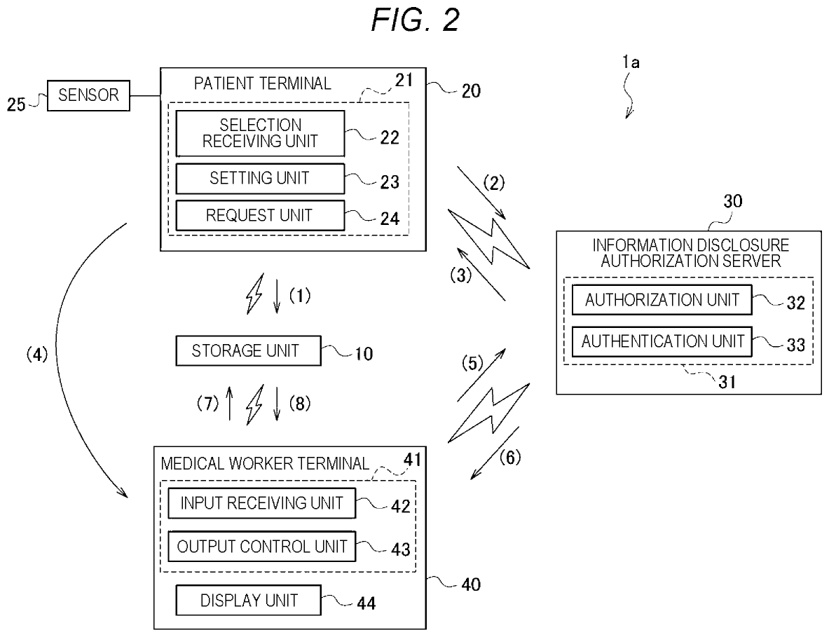

FIG. 2 is a block diagram illustrating an information disclosure system 1a according to the first example.

The information disclosure system 1a includes a storage unit 10, a patient terminal 20, an information disclosure authorization server 30, and a medical worker terminal 40.

### Storage Unit 10

The storage unit 10 is a device that stores information (for example, a server). The storage unit 10 may store patient information transmitted from the patient terminal 20 in correlation with the patient terminal 20 (patient). That is, the storage unit 10 stores patient information including one or more types of details (examples of the types include a disease, a blood pressure, a heart rate, a pulse rate, a number of steps, and a number of smoked cigarettes) pertaining to treatment of a patient. The storage unit 10 may be included as a part of the information disclosure authorization server 30 which will be described later.

### Patient Terminal 20

The patient terminal 20 is a terminal that is used by a patient and is, for example, a smartphone, a tablet, a laptop computer, a personal computer, or a smart watch. The patient terminal 20 stores patient information input by a patient on the basis of operation of an application or acquired on the basis of operation of the sensor 25 in the storage unit 10. That is, when patient information input on the basis of a patient's operation and patient information generated by measuring a patient using the sensor 25 provided in the patient terminal 20 are acquired, the patient terminal 20 transmits the patient information to the storage unit 10 (see (1) in FIG. 2).

The patient terminal 20 includes a selection receiving unit 22, a setting unit 23, and a request unit 24. The selection receiving unit 22, the setting unit 23, and the request unit 24 may be functions of a control unit of the patient terminal 20 (a patient terminal control unit 21) (for example, an arithmetic processing device).

The selection receiving unit 22 receives a selection of details to be disclosed to a medical worker out of the patient information. The medical worker may be, for example, a doctor, a nurse, or another health professional. The selection receiving unit 22 receives a selection of one or more types of details to be disclosed to the medical worker out of the patient information (a plurality of types of details) on the basis of the patient's operation of an input device (not illustrated) of the patient terminal 20. For example, when a plurality of applications are installed on the patient terminal 20, the selection receiving unit 22 receives a selection of details disclosable to the medical worker for each application or each type of details.

That is, for example, the selection receiving unit 22 may receive a selection of enabling disclosure of all the patient information (a plurality of types of details) recorded in one application as a disclosure selection for the application.

Alternatively, for example, the selection receiving unit 22 may receive a selection of one or more types of disclosable details out of the patient information (a plurality of types of details) recorded in one or more applications as a disclosure selection for each type of details. In this example, the selection receiving unit 22 may receive a selection of one or more types of disclosable details out of a plurality of types of details shared by a plurality of applications. The shared details are the same details registered in a plurality of applications.

When a plurality of types of details pertaining to medical treatment of a patient, actions of a patient, and a biological body of a patient are acquired as patient information, the setting unit 23 sets at least one of a plurality of types of details to be disclosable to a medical worker on the basis of the patient's operation. The setting unit 23 sets the details received by the selection receiving unit 22 to be disclosable to a medical worker.

For example, the setting unit 23 may set a flag “1” indicating that details are disclosable for details disclosable to a medical worker (details selected by the selection receiving unit 22) out of the patient information (a plurality of types of details) and set a flag “0” indicating that details are not disclosable for details not disclosable to a medical worker (details selected by the selection receiving unit 22). Disclosability settings of the setting unit 23 are not limited to the example in which a flag is set as described above, but may be performed using various methods as long as the methods can distinguish disclosable details and non-disclosable details for a medical worker.

The request unit 24 generates request information for requesting disclosure of the details of which the selection is received by the selection receiving unit 22. For example, the request unit 24 generates request information for requesting disclosure of the details to request the information disclosure authorization server 30 to authorize disclosure of the patient information (one or more types of details) set to be disclosable by the setting unit 23. In this example, the request unit 24 may generate request information including the details set by the setting unit 23 (setting of the flag for patient information).

The patient terminal 20 transmits the request information generated by the request unit 24 to the information disclosure authorization server 30 via a communication unit (not illustrated) (see (2) in FIG. 2).

### Information Disclosure Authorization Server 30

The information disclosure authorization server 30 authorizes disclosure of patient information. The information disclosure authorization server 30 is, for example, a server that manages patient information and controls and shares information disclosure. Specifically, the information disclosure authorization server 30 includes an authorization unit 32 and an authentication unit 33. The authorization unit 32 and the authentication unit 33 may be functions of a control unit (a server control unit 31) (for example, an arithmetic processing device) of the information disclosure authorization server 30.

The authorization unit 32 generates authorization information for authorizing disclosure of details included in the patient information on the basis of the request information generated by the request unit 24 and provides the authorization information to the patient terminal 20 owned by the patient. That is, the authorization unit 32 generates the authorization information to authorize disclosure of the patient information (one or more types of details) of which the selection is received by the patient terminal 20 (the selection receiving unit 22) to a medical worker. For example, the authorization information may be information of a character string including at least one of characters, numerals, and symbols or may be a two-dimensional information code such as a QR code (registered trademark).

For example, the authorization unit 32 may generate the authorization information in which the patient information (one or more types of details) selected by the selection receiving unit 22, that is, details disclosable to a medical worker, is recorded.

Alternatively, for example, the authorization unit 32 may generate the authorization information including information for authorizing access of the medical worker terminal 40 to the storage unit 10.

In this example, the authorization unit 32 may add deadline information pertaining to a validity term in which the patient information is disclosable to the authorization information. For example, the authorization unit 32 may perform setting such that the patient information (one or more types of details) of which the selection is received by the selection receiving unit 22 can be disclosed to the medical worker terminal 40 (a medical worker) only within a predetermined time (a validity term) after the authorization information has been generated.

The information disclosure authorization server 30 transmits the authorization information generated by the authorization unit 32 to the patient terminal 20 via a communication unit (not illustrated) (see (3) in FIG. 2).

For example, when the authorization information is received, the patient terminal 20 displays a character string or an image (a two-dimensional information code) based on the authorization information.

The authentication unit 33 authorizes disclosure of details included in the patient information of which the selection is received by the selection receiving unit 22 on the basis of the authorization information received by an input receiving unit 42 of the medical worker terminal 40 which will be described later such that acquisition of the details of the patient information from the storage unit 10 is enabled. When the authorization information is received from the medical worker terminal 40 which will be described later, the authentication unit 33 authorizes access of the medical worker terminal 40 (a medical worker) to the patient information stored in the storage unit 10 and enables access of the medical worker terminal 40 to the patient information stored in the storage unit 10 when the authentication has succeeded. In this example, the authentication unit 33 may transmit authentication information indicating that the medical worker terminal 40 can access the patient information stored in the storage unit 10 to the medical worker terminal 40 (see (6) in FIG. 2).

For example, when details disclosable to the medical worker terminal 40 are added to the authorization information by the authorization unit 32, the authentication unit 33 authorizes disclosure of the details in a disclosable range thereof. That is, the authentication unit 33 correlates the authorization information generated by the authorization unit 32 and the authorization information transmitted from the medical worker terminal 40, generates authentication information for authorizing disclosure in the disclosable range of the details included in the authorization information, and transmits the authentication information to the medical worker terminal 40 via a communication unit (not illustrated).

Alternatively, for example, when information for authorizing access of the medical worker terminal 40 to the storage unit 10 is added to the authorization information by the authorization unit 32, the authentication unit 33 correlates the authorization information generated by the authorization unit 32 and the authorization information transmitted from the medical worker terminal 40 and transmits authentication information to which the patient information (one or more types of details) of which the selection is received by the selection receiving unit 22 to the medical worker terminal 40 via a communication unit (not illustrated).

### Medical Worker Terminal 40

The medical worker terminal 40 is a terminal that is used by a medical worker and that includes an output unit. The medical worker terminal 40 is, for example, a laptop computer or a personal computer. The output unit is, for example, a display unit 44 that displays text and an image. The output unit may be a printer (not illustrated) that prints the text and the image.

The medical worker terminal 40 includes an input receiving unit 42, an output control unit 43, and the output unit. The input receiving unit 42 and the output control unit may be functions of a control unit (a medical worker terminal control unit 41) (for example, an arithmetic processing device) of the medical worker terminal 40.

When authorization information provided from the authorization unit 32 to the patient terminal 20 is received (see (4) in FIG. 2), the input receiving unit 42 receives an input of the authorization information.

For example, when a two-dimensional information code (authorization information) displayed on the patient terminal 20 is read using a camera unit (not illustrated), the input receiving unit 42 may receive an input of the authorization information.

Alternatively, for example, when a medical worker inputs authorization information to the medical worker terminal 40 by operating an input device (for example, a keyboard 4 (see FIG. 1)) on the basis of a character string (authorization information) displayed on the patient terminal 20, the input receiving unit 42 receives an input of the authorization information.

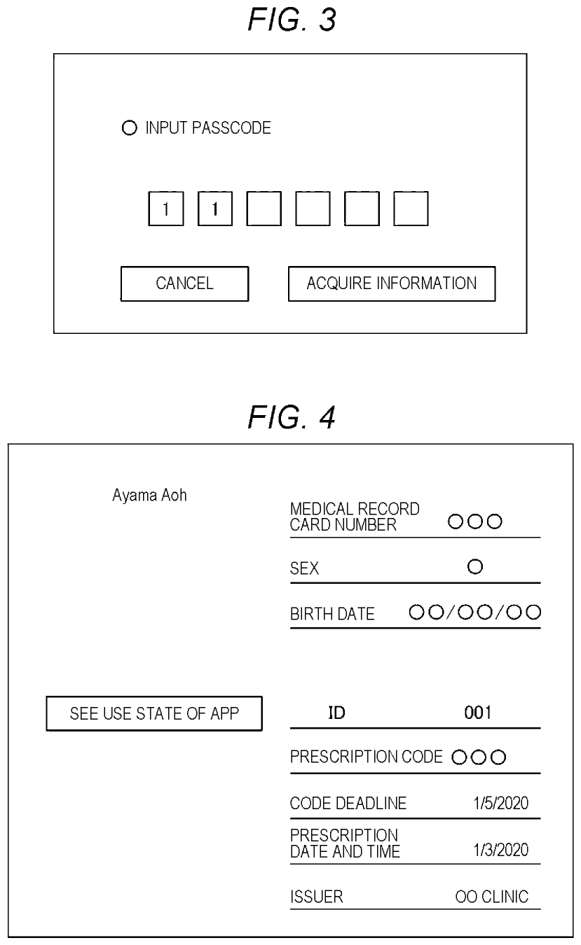

FIG. 3 is a diagram illustrating an example of a screen displayed on a display unit 44 of the medical worker terminal 40 to allow authorization information to be input.

As illustrated in FIG. 3, a screen for inputting a character string as authorization information (a passcode) is displayed on the display unit 44.

A button for reading a two-dimensional information code may be displayed on the screen. When this button is operated, a two-dimensional information code indicating authorization information can be acquired using a camera unit (not illustrated) provided in the medical worker terminal 40.

The medical worker terminal 40 transmits the authorization information received by the input receiving unit 42 to the information disclosure authorization server 30 (the authentication unit 33) via a communication unit (not illustrated) (see (5) in FIG. 2).

When acquisition of patient information is authorized by the authentication unit 33, the output control unit 43 controls the output unit such that the patient information of details selected from the storage unit 10 is acquired and the patient information is output.

That is, the output control unit 43 accesses the storage unit 10 (see (7) in FIG. 2) and acquires the patient information (see (8) in FIG. 2). In this example, the output control unit 43 acquires only patient information within a disclosable range recorded in the authorization information or within a disclosable range recorded in the authentication information, that is, patient information (one or more types of details) of which the selection is received by the selection receiving unit 22.

The output unit may be the display unit 44 or the printer as described above. For example, when authentication information is received from the information disclosure authorization server 30, the output control unit 43 displays the patient information (one or more types of details of which the selection is received by the selection receiving unit 22) acquired from the storage unit 10 on the display unit 44 or prints the patient information using the printer.

FIG. 4 is a diagram illustrating an example of a screen that is displayed on the display unit 44 of the medical worker terminal 40 to acquire patient information.

When the storage unit 10 is accessed on the basis of authentication of the authentication unit 33, the output control unit 43 displays a screen of which an example is illustrated in FIG. 4 on the display unit 44. Various types of information for identifying a patient such as a name, a medical record card number, sex, and a birth date of the patient are displayed on the screen. When a button with “see use state of App” in FIG. 4 is operated, the medical worker can disclose patient information selected by the patient (of which the selection is received by the selection receiving unit 22). Here, “App” refers to an application.

An information disclosure method according to the first example will be described below.

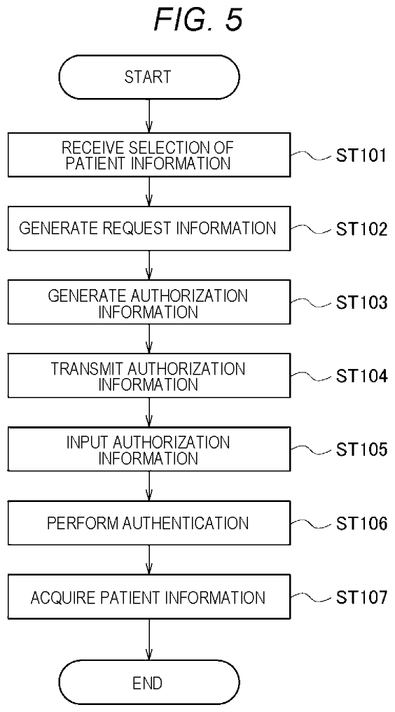

FIG. 5 is a flowchart illustrating the information disclosure method according to the first example.

In Step ST101, the selection receiving unit 22 of the patient terminal 20 receives a selection of details to be disclosed to a medical worker out of patient information. That is, the selection receiving unit 22 receives a unit of disclosure by which the patient information is disclosed to the medical worker.

When a selection of details to be disclosed is received by the selection receiving unit 22, the setting unit 23 of the patient terminal 20 performs setting such that the selected details can be disclosed to the medical worker, for example, by setting a flag indicating whether disclosure is authorized or is not authorized.

In Step ST102, the request unit 24 of the patient terminal 20 generates request information for requesting disclosure of the details of which the selection is received in Step ST101 (details set by the setting unit 23). The patient terminal 20 transmits the generated request information to the information disclosure authorization server 30.

In Step ST103, the authorization unit 32 of the information disclosure authorization server 30 generates authorization information for authorizing disclosure of the details selected in Step ST101 on the basis of the request information generated in Step ST102. The authorization unit 32 transmits the generated authorization information to the patient terminal 20.

In Step ST104, the patient terminal 20 displays the authorization information on the display unit 44 (not illustrated) and transmits the authorization information to the medical worker terminal 40 (the medical worker) when the authorization information transmitted from the information disclosure authorization server 30 in Step ST103 is received.

In Step ST105, the input receiving unit 42 of the medical worker terminal 40 receives an input of the authorization information transmitted in Step ST104.

For example, the input receiving unit 42 may receive an input of the authorization information by reading a two-dimensional information code (authorization information) displayed on the patient terminal 20 using a camera unit (not illustrated) connected to the medical worker terminal 40.

For example, when the medical worker inputs a character string (authorization information) displayed on the patient terminal 20 to the medical worker terminal 40 by operating an input device (for example, a keyboard 4), the input receiving unit 42 receives the input of the authorization information.

When the input of the authorization information is received, the medical worker terminal 40 transmits the authorization information to the information disclosure authorization server 30.

In Step ST106, the authentication unit 33 of the information disclosure authorization server 30 authorizes disclosure of details included in the patient information of which the selection is received in Step ST101 on the basis of the authorization information received in Step ST105 such that acquisition of the details of the patient information from the storage unit 10 is enabled. That is, the authentication unit 33 authorizes access of the medical worker terminal 40 (the medical worker) to the patient information stored in the storage unit 10 when the authorization information is received from the medical worker terminal 40, and enables access of the medical worker terminal 40 to the patient information stored in the storage unit 10 when the authentication has succeeded. That is, the authentication unit 33 transmits authentication information for enabling access of the medical worker terminal 40 to the patient information stored in the storage unit 10 to the medical worker terminal 40.

In Step ST107, when it is determined in Step ST106 that the authentication has succeeded, the output control unit 43 acquires patient information of only the details selected in Step ST101 from the storage unit 10 and performs control such that the acquired patient information is output. For example, the output control unit 43 controls the display unit 44 such that the acquired patient information is displayed.

In this example, the storage unit 10 may perform authentication of access of the medical worker terminal 40 thereto based on the authentication information. For example, when authentication information transmitted from the medical worker terminal 40 is received, the storage unit 10 inquires the information disclosure authorization server 30 about whether the authentication information is right. When the information disclosure authorization server 30 determines that the authentication information is right, the storage unit 10 enables the medical worker terminal 40 to read the patient information.

With the information disclosure system 1a according to the first example, for example, the following advantages can be achieved.

That is, since the information disclosure system 1a allows a patient to select one or more types of details out of patient information acquired from a patient terminal 20 and allows a medical worker terminal 40 (a medical worker) to disclose only the details selected by the patient on the basis of authorization and authentication of the information disclosure authorization server 30, it is possible to perform protection pertaining to disclosure of details acquired from the patient terminal 20.

With the information disclosure method, it is possible to achieve the same advantages as in the information disclosure system 1a.

The constituent units of the information disclosure system 1a (the patient terminal 20, the information disclosure authorization server 30, and the medical worker terminal 40) may be functions of an arithmetic processing device or the like of a computer. That is, the selection receiving unit 22, the setting unit 23, the request unit 24, the authorization unit 32, the authentication unit 33, the input receiving unit 42, and the output control unit 43 of the information disclosure system 1a (the patient terminal 20, the information disclosure authorization server 30, and the medical worker terminal 40) may be a selection receiving function, a setting function, a request function, an authorization function, an authentication function, an input receiving function, and an output control function of an arithmetic processing device or the like of a computer.

An information disclosure program can cause computers (the patient terminal 20, the information disclosure authorization server 30, and the medical worker terminal 40) to embody the aforementioned functions. The information disclosure program may be recorded in a non-transitory computer-readable recording medium such as an external memory or an optical disc.

As described above, the constituent units of the information disclosure system 1a (the patient terminal 20, the information disclosure authorization server 30, and the medical worker terminal 40) may be an arithmetic processing device or the like of a computer. The arithmetic processing device or the like is constituted, for example, by an integrated circuit. Accordingly, the constituent units of the information disclosure system 1a (the patient terminal 20, the information disclosure authorization server 30, and the medical worker terminal 40) may be circuits constituting the arithmetic processing device or the like. That is, the selection receiving unit 22, the setting unit 23, the request unit 24, the authorization unit 32, the authentication unit 33, the input receiving unit 42, and the output control unit 43 of the information disclosure system 1a (the patient terminal 20, the information disclosure authorization server 30, and the medical worker terminal 40) may be a selection receiving circuit, a setting circuit, a request circuit, an authorization circuit, an authentication circuit, an input receiving circuit, and an output control circuit of an arithmetic processing device or the like of a computer.

The sensor 25 that is connected to the patient terminal 20 and the display unit 44 (the output unit) of the medical worker terminal 40 may be, for example, a sensor function and a display function (an output function) of the functions of the arithmetic processing device or the like. The sensor 25 and the display unit 44 (the output unit) may be a sensor circuit and a display circuit (an output circuit) constituted, for example, by an integrated circuit. The sensor 25 and the display unit 44 (the output unit) may be a sensor device and a display device (an output device) including, for example, a plurality of devices.

### Second Example

A second example will be described below. The second example is characterized in that a patient terminal 200 can perform setting of details to be disclosed to a medical worker in a list included in request information in addition to features of the first example.

Parts of the second example different from those of the first example will be mainly described, and description of parts common or similar to those of the first example will be omitted or simplified.

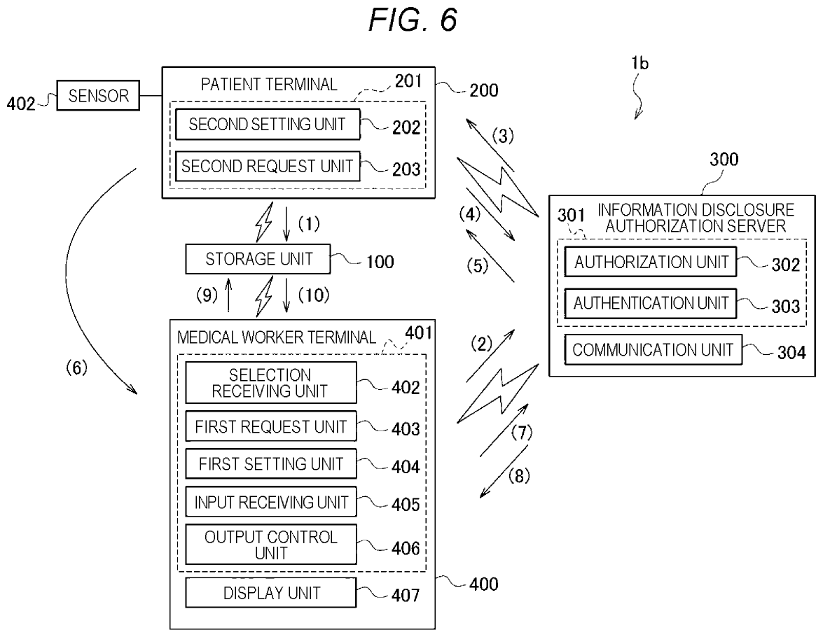

FIG. 6 is a block diagram illustrating an information disclosure system 1b according to the second example.

The information disclosure system 1b includes a storage unit 100, a patient terminal 200, an information disclosure authorization server 300, and a medical worker terminal 400.

### Storage Unit 100

The storage unit 100 stores patient information including one or more types of details pertaining to treatment of a patient.

### Medical Worker Terminal 400

The medical worker terminal 400 includes a selection receiving unit 402, a first request unit 403, a first setting unit 404, an input receiving unit 405, an output control unit 406, and an output unit. For example, the output unit may be a display unit 44 that displays text and an image or may be a printer (not illustrated) that prints text and an image. The selection receiving unit 402, the first request unit 403, the first setting unit 404, the input receiving unit 405, and the output control unit 406 may be functions of a control unit of the medical worker terminal 400 (a medical worker terminal control unit 401) (for example, an arithmetic processing device).

The selection receiving unit 402 receives a selection of details to be disclosed to a medical worker out of patient information. The selection receiving unit 402 receives a selection of details to be disclosed to a medical worker out of patient information of a specific patient on the basis of the medical worker's operating an input device (for example, a keyboard 4 (see FIG. 1)) of the medical worker terminal 400.

The first request unit 403 generates first request information for requesting disclosure of details of which the selection is received by the selection receiving unit 402. The first request unit 403 may generate first request information including setting of a flag when the flag is set by the first setting unit 404 provided in the medical worker terminal 400 according to the second example similarly to the setting unit 23 according to the first example.

The medical worker terminal 400 transmits the first request information to the information disclosure authorization server 300 (see (2) in FIG. 6).

When authorization information provided from an authorization unit 302 of the information disclosure authorization server 300 which will be described later to the patient terminal 200 is received (see (6) in FIG. 6), the input receiving unit 405 receives an input of the authorization information.

For example, when a two-dimensional information code (authorization information) displayed on the patient terminal 200 is read using a camera unit (not illustrated), the input receiving unit 405 may receive an input of the authorization information.

Alternatively, for example, when a medical worker inputs authorization information to the medical worker terminal 400 by operating an input device (for example, a keyboard 4 (see FIG. 1)) on the basis of a character string (authorization information) displayed on the patient terminal 200, the input receiving unit 405 receives an input of the authorization information.

The medical worker terminal 400 transmits authorization information received by the input receiving unit 405 to the information disclosure authorization server 300 (an authentication unit 303 which will be described later) (see (7) in FIG. 6).

When acquisition of patient information is authorized by an authentication unit 303 of the information disclosure authorization server 300 which will be described later, the output control unit 406 controls the output unit such that details authorized by a patient (patient information) is acquired from the storage unit 100 and the patient information is output. The output control unit 406 accesses the storage unit 100 (see (9) in FIG. 6) and acquires the patient information (see (10) in FIG. 6). That is, when the authentication information transmitted from the information disclosure authorization server 300 is received, the output control unit 406 acquires only patient information (one or more types of details) in a range of which disclosure is authorized by the patient terminal 200 (the patient) from the storage unit 100. For example, the output control unit 406 displays the patient information (one or more types of details) acquired from the storage unit 100 on the display unit 407 or prints the patient information using a printer (not illustrated).

### Information Disclosure Authorization Server 300

The information disclosure authorization server 300 includes an authorization unit 302, an authentication unit 303, and a communication unit 304. The communication unit 304 corresponds to an example of a “transmission unit” in the appended claims. The authorization unit 302 and the authentication unit 303 may be functions of a control unit (a server control unit 301) (for example, an arithmetic processing device) of the information disclosure authorization server 300.

The communication unit 304 generates request information for requesting a patient to disclose patient information on the basis of the first request information generated by the first request unit 403 of the medical worker terminal 400 and transmits the generated request information to the patient terminal 200 under the control of the server control unit 301 (see (3) in FIG. 6). The request information is information for requesting a patient to ascertain whether the patient information (one or more types of details) of which the selection is received by the selection receiving unit 402 of the medical worker terminal 400 is to be disclosed to a medical worker. For example, the request information may include a list of patient information (one or more types of details) of which the selection is received by the selection receiving unit 402 or the like.

The authorization unit 302 generates authorization information for authorizing disclosure of details included in the patient information on the basis of second request information generated by a second request unit 203 of the patient terminal 200 which will be described later and provides the authorization information to the patient terminal 200 owned by the patient. That is, the authorization unit 302 generates the authorization information to authorize disclosure of the details of which disclosure is authorized by the patient (details which are set to “disclosable” by a second setting unit 202) to a medical worker. For example, the authorization information may be information of a character string or a two-dimensional information code. In this example, the authorization unit 302 may add deadline information pertaining to a validity term in which the patient information is disclosable to the authorization information.

The information disclosure authorization server 300 transmits the authorization information generated by the authorization unit 302 to the patient terminal 200 (see (5) in FIG. 6).

For example, when the authorization information is received, the patient terminal 200 displays a character string or an image (a two-dimensional information code) based on the authorization information.

The authentication unit 303 authorizes disclosure of details included in the patient information of which the selection is received by the selection receiving unit 402 on the basis of the authorization information received by an input receiving unit 405 of the medical worker terminal 400 such that acquisition of the details of the patient information from the storage unit 100 is enabled. That is, when the authorization information is received from the medical worker terminal 400, the authentication unit 303 authorizes access of the medical worker terminal 400 (a medical worker) to the patient information stored in the storage unit 100 and transmits authentication information for enabling access of the medical worker terminal 400 to the patient information stored in the storage unit 100 when the authentication has succeeded to the medical worker terminal 400 (see (8) in FIG. 6).

The authentication unit 303 does not permit disclosure of patient information (one or more types of details) of which disclosure is rejected by the patient (the patient terminal 200) out of patient information (one or more types of details) of which the selection is received by the selection receiving unit 402. That is, the authentication unit 303 permits disclosure of details set by the second setting unit 202 of the patient terminal 200 which will be described later on the basis of the authorization information received by the input receiving unit 405 and enables acquisition of the details of patient information from the storage unit 100.

### Patient Terminal 200

When patient information input on the basis of a patient's operation and patient information generated by measuring a patient using a sensor 204 provided in the patient terminal 200 are acquired, the patient terminal 200 transmits the patient information to the storage unit 100 (see (1) in FIG. 6). The patient terminal 200 includes a second setting unit 202 and a second request unit 203. The second setting unit 202 and the second request unit 203 may be functions of a control unit of the patient terminal 200 (a patient terminal control unit 201) (for example, an arithmetic processing device).

When a plurality of types of details pertaining to medical treatment of a patient, actions of a patient, and a biological body of a patient are acquired as patient information, the second setting unit 202 sets at least one of the plurality of types of details to be disclosable to a medical worker on the basis of the patient's operation. That is, the second setting unit 202 sets details to be disclosable to a medical worker in a list included in the request information. For example, the second setting unit 202 may set details not to be disclosable even when the details are requested to be disclosed by the medical worker. In this example, the second setting unit 202 may change a flag in the list included in the request information or may not change the flag.

The second request unit 203 acquires request information transmitted to the patient terminal 200 by the communication unit 304 of the information disclosure authorization server 300, generates second request information for requesting disclosure of patient information based on the request information, and transmits the second request information to the information disclosure authorization server 300 (see (4) in FIG. 6). That is, the second request unit 203 generates second request information for requesting disclosure of details set by the second setting unit 202 to a medical worker and transmits the second request information to the information disclosure authorization server 300.

An information disclosure method according to the second example will be described below.

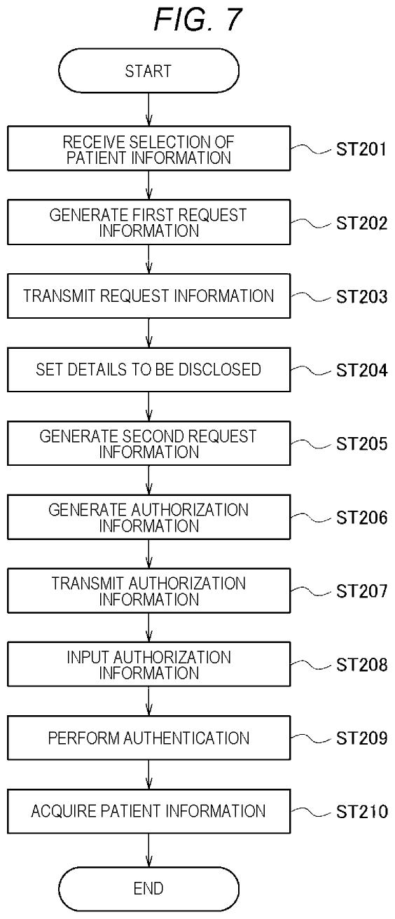

FIG. 7 is a flowchart illustrating the information disclosure method according to the second example.

In Step ST201, the selection receiving unit 402 of the medical worker terminal 400 receives a selection of details to be disclosed to a medical worker out of patient information. For example, the first setting unit 404 may set different flags for details of which disclosure is requested and details of which disclosure is not requested on the basis of the details received by the selection receiving unit 402.

In Step ST202, the first request unit 403 of the medical worker terminal 400 generates first request information for requesting disclosure of the details of which the selection is received in Step ST201. The medical worker terminal 400 transmits the generated first request information to the information disclosure authorization server 300.

In Step ST203, the communication unit 304 of the information disclosure authorization server 300 generates request information for requesting a patient to disclosure patient information on the basis of the first request information generated in Step ST202 and transmits the request information to the patient terminal 200 under the control of the server control unit 301.

In Step ST204, the second setting unit 202 of the patient terminal 200 sets patient information (one or more types of details) of which disclosure to a medical worker is permitted on the basis of the request information transmitted in Step ST203.

In Step ST205, the second request unit 203 of the patient terminal 200 generates second request information for requesting disclosure of the details set in Step ST204 to a medical worker and transmits the second request information to the information disclosure authorization server 300.

In Step ST206, the authorization unit 302 of the information disclosure authorization server 300 generates authorization information of authorizing disclosure of details included in the patient information on the basis of the second request information transmitted in Step ST205 and provides the authorization information to the patient terminal 200 owned by the patient.

In Step ST207, when the authorization information transmitted from the information disclosure authorization server 300 in Step ST206 is received, the patient terminal 200 displays the authorization information on a display unit (not illustrated) and notifies the medical worker terminal 400 (the medical worker).

In Step ST208, the input receiving unit 405 of the medical worker terminal 400 receives an input of the authorization information transmitted in Step ST207.

When the input of the authorization information is received, the medical worker terminal 400 transmits the authorization information to the information disclosure authorization server 300.

In Step ST209, the authentication unit 303 of the information disclosure authorization server 300 performs authentication on the basis of the authorization information received in Step ST208, and authorizes disclosure of the patient information (one or more types of details) set in Step ST208 such that acquisition of the details of the patient information from the storage unit 100 is enabled when the authentication has succeeded. That is, the authentication unit 303 transmits authentication information for enabling access of the medical worker terminal 400 to the patient information stored in the storage unit 100 to the medical worker terminal 400.

In Step ST210, when the authentication has succeeded in Step ST209, the output control unit 406 acquires only patient information (one or more types of details of which disclosure is permitted by the patient) set in Step ST204 from the storage unit 100 and performs control such that the patient information is output. For example, the output control unit 406 controls the display unit 407 such that the acquired patient information is displayed.

With the information disclosure system 1b according to the second example, for example, the following advantages can be achieved.

That is, in the information disclosure system 1b, when a request for disclosure of patient information (one or more types of details) of a specific patient is transmitted from the medical worker terminal 400, the information disclosure authorization server 300 requests the patient terminal 200 to ascertain whether details requested for disclosure are to be disclosed to a medical worker, and the information disclosure authorization server 300 permits disclosure of only one or more types of details of which disclosure is authorized by a patient (the patient terminal 200) out of one or more types of details of which disclosure is requested by the medical worker terminal 400 to the medical worker terminal 400. Accordingly, it is possible to perform protection pertaining to disclosure of details acquired by the patient terminal 200.

With the information disclosure method, it is possible to achieve the same advantages as in the information disclosure system 1b.

The constituent units of the information disclosure system 1b (the patient terminal 200, the information disclosure authorization server 300, and the medical worker terminal 400) may be functions of an arithmetic processing device or the like of a computer. That is, the second setting unit 202, the second request unit 203, the authorization unit 302, the authentication unit 303, the selection receiving unit 402, the first request unit 403, the first setting unit 404, the input receiving unit 405, and the output control unit 406 of the information disclosure system 1b (the patient terminal 200, the information disclosure authorization server 300, and the medical worker terminal 400) may be a second setting function, a second request function, an authorization function, an authentication function, a selection receiving function, a first request function, a first setting function, an input receiving function, and an output control function of an arithmetic processing device or the like of a computer.

An information disclosure program can cause computers (the patient terminal 200, the information disclosure authorization server 300, and the medical worker terminal 400) to embody the aforementioned functions. The information disclosure program may be recorded in a non-transitory computer-readable recording medium such as an external memory or an optical disc.

As described above, the constituent units of the information disclosure system 1b (the patient terminal 200, the information disclosure authorization server 300, and the medical worker terminal 400) may be an arithmetic processing device or the like of a computer. The arithmetic processing device or the like is constituted, for example, by an integrated circuit. Accordingly, the constituent units of the information disclosure system 1b (the patient terminal 200, the information disclosure authorization server 300, and the medical worker terminal 400) may be circuits constituting the arithmetic processing device or the like. That is, the second setting unit 202, the second request unit 203, the authorization unit 302, the authentication unit 303, the selection receiving unit 402, the first request unit 403, the first setting unit 404, the input receiving unit 405, and the output control unit 406 of the information disclosure system 1b (the patient terminal 200, the information disclosure authorization server 300, and the medical worker terminal 400) may be a second setting circuit, a second request circuit, an authorization circuit, an authentication circuit, a selection receiving circuit, first request circuit, a first setting circuit, an input receiving circuit, and an output control circuit of an arithmetic processing device or the like of a computer.

The sensor 204 that is connected to the patient terminal 200, the communication unit 304 of the information disclosure authorization server 300, and the display unit 407 (the output unit) of the medical worker terminal 400 may be, for example, a sensor function, a communication function, and a display function (an output function) of the functions of the arithmetic processing device or the like. The sensor 204, the communication unit 304, and the display unit 407 (the output unit) may be a sensor circuit, a communication circuit, and a display circuit (an output circuit) constituted, for example, by an integrated circuit. The sensor 204, the communication unit 304, and the display unit 407 (the output unit) may be a sensor device, a communication device, and a display device (an output device) including, for example, a plurality of devices.

What is claimed is:

1. An information disclosure system including a patient terminal used by a patient, an information disclosure authorization server that authorizes information disclosure, and a medical worker terminal used by a medical worker and includes an output unit, the information disclosure system comprising:
a selection receiving unit provided in the patient terminal and configured to receive a selection of details to be disclosed to the medical worker out of patient information including one or more types of details pertaining to treatment of the patient;
a request unit provided in the patient terminal and configured to generate request information for requesting disclosure of the details of which the selection is received by the selection receiving unit;
an authorization unit provided in the information disclosure authorization server and configured to generate authorization information for authorizing disclosure of details included in the patient information based on the request information generated by the request unit and to provide the generated authorization information to the patient terminal owned by the patient;
an input receiving unit provided in the medical worker terminal and configured to receive an input of the authorization information when the authorization information provided from the authorization unit to the patient terminal is received;
an authentication unit provided in the information disclosure authorization server and configured to authorize disclosure of details included in the patient information of which the selection is received by the selection receiving unit based on the authorization information received by the input receiving unit and to enable acquisition of the details of the patient information; and
an output control unit provided in the medical worker terminal and configured to control the output unit such that the patient information is output when acquisition of the patient information is authorized by the authentication unit.

2. The information disclosure system according to claim 1, wherein the authorization unit is configured to add deadline information pertaining to a validity term in which disclosure of patient information is disclosable to the authorization information.

3. The information disclosure system according to claim 1, further comprising a storage unit configured to store patient information,
wherein the authentication unit is configured to enable acquisition of the details of the patient information from the storage unit, and
the output control unit is configured to acquire the details of the patient information from the storage unit and to output the patient information.

4. The information disclosure system according to claim 3, wherein the patient terminal transmits patient information to the storage unit when patient information which is input on the basis of the patient's operation and patient information generated by measuring the patient using a sensor provided in the patient terminal are acquired.

5. The information disclosure system according to claim 4, wherein the patient terminal includes a setting unit configured to set at least one of a plurality of types of details pertaining to medical treatment of a patient, actions of a patient, and a biological body of a patient as the patient information such that the at least one is disclosable to the medical worker on the basis of the patient's operation when the plurality of types of details are acquired.

6. The information disclosure system according to claim 5, wherein the authorization unit is configured to add deadline information pertaining to a validity term in which patient information is disclosable to the authorization information.

7. The information disclosure system according to claim 5, wherein the patient terminal transmits patient information to the storage unit when patient information which is input on the basis of the patient's operation and patient information generated by performing measurement of the patient using a sensor provided in the patient terminal are acquired.

8. A server connected to a patient terminal used by a patient and a medical worker terminal used by a medical worker via a communication network and that authorizes information disclosure, the server comprising:
a communication unit configured to receive request information for requesting disclosure of selected details to be disclosed to the medical worker transmitted from the patient terminal when a selection of the details out of patient information including one or more types of details pertaining to treatment of the patient is received by the patient terminal;
an authorization unit configured to generate authorization information for authorizing disclosure of the details included in the patient information based on the request information received by the communication unit and to provide the generated authorization information to the patient terminal owned by the patient; and
an authentication unit configured to authorize disclosure of details included in the patient information of which the selection is received by the patient terminal based on the authorization information and to enable acquisition of the details of the patient information from a storage unit in which the details of the patient information are stored when the authorization information provided to the patient terminal is received by the medical worker terminal and the authorization information transmitted from the medical worker terminal is received by the communication unit.

9. An information disclosure method performed by an information disclosure system including a patient terminal used by a patient, an information disclosure authorization server that authorizes information disclosure, and a medical worker terminal used by a medical worker and includes an output unit, the information disclosure method comprising:
a selection receiving step of causing the patient terminal to receive a selection of details to be disclosed to the medical worker out of patient information;
a request step of causing the patient terminal to generate request information for requesting disclosure of the details of which the selection is received in the selection receiving step;
an authorization step of causing the information disclosure authorization server to generate authorization information for authorizing disclosure of details included in the patient information based on the request information generated in the request step and to provide the generated authorization information to the patient terminal owned by the patient;
an input receiving step of causing the medical worker terminal to receive an input of the authorization information when the authorization information provided to the patient terminal in the authorization step is received;
an authentication step of causing the information disclosure authorization server to authorize disclosure of details included in the patient information of which the selection is received in the selection receiving step based on the authorization information received in the input receiving step and to enable acquisition of the details of the patient information; and
an output control step of causing the medical worker terminal to acquire the details of the patient information and to control the output unit such that the patient information is output when acquisition of the patient information is authorized in the authentication step.

10. The information disclosure system according to claim 2, further comprising a storage unit configured to store patient information,
wherein the authentication unit is configured to enable acquisition of the details of the patient information from the storage unit, and
the output control unit is configured to acquire the details of the patient information from the storage unit and to output the patient information.
